(19) United States
(12) Patent Application Publication
(10) Pub. No.: US 2004/0038270 A1
(43) Pub. Date: Feb. 26, 2004

(54) In situ hybridization arrangement for the specific detection of microorganisms

(76) Inventors: Peter Muhlhahn, Munchen (DE); Jiri Snaidr, Munchen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/458,775

(22) Filed: Jun. 10, 2003

Related U.S. Application Data
(63) Continuation of application No. PCT/EP01/14543, filed on Dec. 11, 2001.

(30) Foreign Application Priority Data
Dec. 11, 2000 (DE) 100 61 655.0

(51) Int. Cl. C12Q 1/68; C12M 1/34

(52) U.S. Cl. 435/6; 435/287.2

(57) ABSTRACT

The present invention describes an in situ hybridization arrangement for the specific detection of microorganisms in a sample. Such arrangement has a container with at least one opening, a support for the hybridization solution, a slide, and a fastening means for the slide. The invention further describes a method for specific detection of microorganisms in a sample by in situ hybridization. The method of the present invention comprises of the steps of: fixing the microorganisms contained in the sample; incubating the fixed cells with detectable nucleic acid probe molecules; removing or washing-off the non-hybridized nucleic acid probe molecules, and detecting the cells hybridized with the nucleic acid probe molecules. Such method is carried out using the in situ hybridization arrangement of the invention. The invention also encompasses a kit for a specific detection of microorganisms by in situ hybridization using the in situ hybridization arrangement of the invention

### RELATED APPLICATIONS

This Application is a continuation of International Application PCT/EP01/14543, filed Dec. 11, 2001 and published in German as WO 02/48398 A2 on June 2002, which claims the benefit of priority to German Application 100 61 655.0, filed Dec. 11, 2000, both of which are incorporated herein by reference in their entireties.

### FIELD OF THE INVENTION

The invention relates to an in situ hybridization arrangement for the specific detection of microorganisms, a method for specific detection of microorganisms by in situ hybridization and a kit, which permits the identification and visualization of microorganisms in a sample.

### BACKGROUND OF THE INVENTION

All current methods for determining bacteria have a common objective: they attempt to circumvent the disadvantages of cultivation by eliminating the necessity of cultivation of the bacteria.

In PCR, the polymerase chain reaction, a specific characteristic segment of the bacterial genome is amplified with bacteria-specific primers. If the primer finds its target site, millions of amplicons of a segment of the genetic information are generated. In the subsequent analysis by an agarose gel in order to separate DNA fragments, a qualitative evaluation can be conducted. In the simplest case, this results in the information that the target sites are present in the analyzed sample. Other conclusions are not allowed, since the target sites may be derived from a living bacterium, a dead bacterium or from naked DNA. Here, differentiation is not possible. A further development of this technique is quantitative PCR, in which it is attempted to generate a correlation between the amount of bacteria present and the amount of DNA obtained and amplified.

However, biochemical parameters are also used for identification of bacteria. The generation of bacteria profiles based on quinone analyses serves to reflect the bacterial population with as little bias as possible (Hiraishi, A., Respiratory quinone profiles as tools for identifying different bacterial populations in activated sludge, J. Gen. Appl. Microbiol. (1988) 34:39-56). However, this method as well depends on the cultivation of individual bacteria, since the quinone profiles of the monocultured bacteria are required for generating the reference data-base. In addition, the determination of the bacterial quinone profiles cannot give a real impression of the population distributions actually present in the sample.

In contrast to this, the detection of bacteria by antibodies is a more direct method (Brigmon, R. L., G. Bitton, S. G. Zam, and B. O'Brien, Development and application of a monoclonal antibody against Thiothrix spp., Appl. Environ. Microbiol. (1995) 61:13-20). Fluorescence-labeled antibodies are mixed with the sample and allow highly specific binding to the bacterial antigens. In the epifluorescence microscope, the bacteria are detected subsequently by their emitted fluorescence. In this way, bacteria can be identified down to strain level. However, three critical disadvantages restrict the application of this method: firstly, mono-cultures are required for the production of the antibodies. Secondly, the antibody-fluorescent molecule-complex is often large in volume and unwieldy, which generates problems in entering the target cells. Thirdly, the detection is often too specific. The antibodies are expensive to produce and frequently detect only one specific bacterial strain, but are unable to detect other strains of the same bacterial species. Frequently, however, strain-specific detection of bacteria is not necessary, but instead detection of a bacterial species or an entire bacteria group is required. Fourthly, production of the antibodies is a relatively tedious and expensive procedure.

A unique approach to combine the specificity of the molecular biological methods such as PCR with the possibility to visualize bacteria as represented by the antibody method, is the method of fluorescent in situ hybridization (FISH; Amann, R. I., W. Ludwig, and K.-H. Schleifer, Phylogenetic identification and in situ detection of individual microbial cells without cultivation. Microbiol. Rev. (1995) 59:143-169). Hereby, bacterial species, genera or groups can be visualized and identified highly specifically, directly in the sample. This method is the only approach that gives an unbiased reflection of the actual in situ distributions of the biocoenosis. Even bacteria that have not been cultured until now, and have therefore not been characterized, can be identified and also be visualized directly in the sample.

The FISH technique is based on the fact that there are certain molecules present in bacterial cells, which due to their vital function have been mutated only to a small degree in the course of evolution: the 16S and the 23S ribosomal ribonucleic acid (rRNA). Both are constituents of the ribosomes, the sites of protein biosynthesis, and can serve as phylogenetic markers, due to their ubiquitous distribution, their size and their structural and functional constancy (Woese, C. R., Bacterial evolution, Microbiol. Rev. (1987) 51:221-271). Based on a comparative sequence analysis, phylogenetic relations can be derived solely from these data. For this, these sequence data have to be aligned. In an alignment, which is based on knowledge of the secondary and tertiary structures of these macromolecules, the homologous positions of the ribosomal nucleic acids are correlated. FIG. 1 shows the secondary structure model of a 16S rRNA.

Based on these data, phylogenetic calculations can be performed. Application of state-of-the-art computer technology allows fast and efficient calculations, even if they are large-scale, as well as the establishment of large databases containing the aligned sequences of the 16S rRNA and 23S rRNA. Through fast access to this data material, newly obtained sequences can be analyzed phylogenetically in a short period of time. These rRNA databases can be used to construct specific gene probes. Hereby, all available rRNA sequences are compared and probes are designed for certain sequence parts, which specifically detect a bacterial species, genus or group.

In FISH (fluorescence in situ hybridization), these gene probes, which are complementary to a certain region on the ribosomal target sequence, are introduced into the cell. Usually, the gene probes are small, 16-20 bases long, single-stranded desoxyribonucleic acid fragments, and are directed to a target region, which is typical for a bacterial species or a bacterial group. If the fluorescence-labeled gene probe finds its target sequence in a bacterial cell, so it binds thereto, and the cells can be detected due to their fluorescence in the fluorescence microscope. FIG. 2 illustrates the procedure of in situ hybridization.

Culture-dependent methods give only a very biased insight into the composition and dynamics of the microbial biocoenosis. Using the FISH technique it could be demonstrated that, for example, in detecting activated sludge flora, cultivation results in a cultivation shift (Wagner, M., R. Amann, H. Lemmer, and K. H. Schleifer, Probing activated sludge with oligonucleotides specific for proteobacteria: inadequacy of culture-dependent methods for describing microbial community structure, Appl. Environ. Microbiol. (1993) 59:1520-1525).

By this medium-dependent biasing of the real bacterial community structures, the importance of bacteria that play a subordinate role in activated sludge but have adapted well to the used cultivation conditions, is dramatically overestimated. Thus it could be demonstrated that due to such a cultivation artifact, the bacterial genus Acinetobacter has been completely incorrectly evaluated regarding its role as a biological phosphate remover in waste water treatment. As a result of such erroneous evaluations, cost-intensive, flawed or imprecise plants are designed. The efficiency and reproducibility of such simulation calculations is small.

The advantages of the FISH technique compared to the identification of bacteria using cultivation are manifold. Firstly, many more cells can be detected using gene probes. Whereas maximally only 15% of the bacterial population of a sample can be visualized by cultivation, FISH allows detection of up to 100% of the total bacterial population in many samples. Secondly, the active part of community can be determined by the ratio between the probe, which is directed to all bacteria and an unspecific cell staining. Thirdly, the bacteria are made visible directly in situ (on the spot). Thus, possible interactions between various bacterial populations can be recognized and analyzed. Fourthly, the detection of bacteria using the FISH technique is much faster than using cultivation. Whereas identification of bacteria using cultivation frequently requires several days, the time from taking a sample to identifying the bacteria, even on the species level, takes only a few hours using the FISH technique. Fifthly, gene probes can be selected almost without restriction with regard to their specificity. Individual species can be detected with one probe as well as an entire genera or bacterial groups. Sixthly, bacterial species or entire bacterial populations can be exactly quantified directly in the sample. Cultivation and the associated insufficient quantification are not necessary.

When a bacterium present in a sample is examined taxonomically, the top-to-bottom approach is employed. Hereby, the bacterial sample is analyzed initially with gene probes, whose specificity is as broad as possible, i.e. the specificity is small and detects only entire bacteria groups. A successive increase in the specificity of the probes used eventually leads to the identification of the unknown bacterium.

Thus, the FISH technique is a superior tool for fast and highly specific detection of bacteria, directly in a sample. In contrast to cultivation methods, it is a direct procedure and allows, in contrast to modern methods, not only the visualization of the bacteria but in addition their exact quantification.

In principle, the FISH analysis is performed on a slide, since the bacteria are visualized during evaluation by radiation with high-energy light. The composition of the individual solutions such as hybridization buffer or hybridization solution and washing buffer or washing solution is well known to the expert and is described in detail, for example, in Snaidr et al. (J. Snaidr, R. Amann, I. Huber, W. Ludwig, and K.-H. Schleifer, Phylogenetic analysis and in situ identification of bacteria in activated sludge. Appl. Env. Microb. (1997) 63:7, 2884-2896).

The FISH procedure for the analysis of microorganisms on a slide usually comprises the following steps:

1. Introducing an aliquot of the microbial sample into a reaction vial and mixing it with a suitable fixation solution.

2. Several centrifugation and washing steps until the sample is fixed and becomes accessible for gene probes.

3. Applying an aliquot of the fixed microbial sample into a well on the slide.

4. Drying of the microorganisms on the slide by incubation in an oven at 40-90° C. for 10-30 min.

5. Dehydrating the microbial cells with increasing concentrations of ethanol: hereby the slide is sequentially immersed in solutions with 50%,70% and 100% ethanol.

6. Applying a hybridization solution onto the well containing the microorganisms.

7. Applying a probe solution onto the same well.

8. Preparation of a humid chamber: for this, a piece of cellulose is folded and inserted into a plastic tube. The cellulose, which lies in the chamber, is then moistened with several ml of the hybridization solution.

9. The slide is put horizontally on the cellulose in the humid chamber.

10. The humid chamber is transferred into an incubation oven and incubated for 1-2 hours.

11. The humid chamber is opened; the slide is removed and rinsed briefly with distilled water.

12. The humid chamber is discarded.

13. A washing buffer solution is filled into a new plastic tube.

14. The rinsed slide is inserted into the plastic tube filled with the washing solution.

15. The slide is incubated in the plastic tube in an incubation oven for 10-30 min.

16. The slide is removed from the plastic tube and is washed with distilled water.

17. The slide is tilted and air-dried.

18. After application of an anti-fading reagent onto the slide, the slide can be viewed under an epifluorescence microscope.

However, the above described conventional FISH method for detection of microorganisms is associated with substantial disadvantages. It is elaborate and tedious, and cannot be reproduced with consistent quality due to several sources of error. This is described in detail in the following.

Due to the numerous washing and centrifugation steps, the fixation of the sample can take place with varying efficiency. The result of this is that in the subsequent hybridization step, the gene probes penetrate the cells with varying efficiency, and varying degrees of brightness are the result during detection of the cells in the epifluorescence microscope. Causatively, however, the brightness correlates with the ribosome content of the cells. Therefore, the intensity of the fluorescence, as for in example in FISH analysis, is a measure to determine-whether the growth condition of the cells was good or poor at the time the sample was taken. This information is critical for an overall evaluation of the microbial condition of a sample, especially in medical microbiology, but also in food or environmental microbiology. Varying efficiency in fixation of the sample to be analyzed thus results in biased information about the growth condition and therefore about the overall condition of a sample.

Furthermore, a poor signaling intensity due to inefficient fixation diminishes the ability of the examiner to also detect small cells or cells with a low ribosomal content during visualization in the epifluorescence microscope. In addition, cells may be lost during the fixation process in reaction vials due to the various washing and centrifugation steps.

Another problem of the conventional FISH method is that cells can detach from the slide or be transferred to other wells during dehydration of the cells during several incubations.

Furthermore, the separation of probe solution and hybridization solution results in higher working expenditure, as two different solutions must be applied to the slide well.

Furthermore, the preparation of the humid chamber is inconvenient and does not guarantee a horizontal position of the slide. This may result in mixing of the different solutions present in the different wells.

Another problem in using a round plastic tube as hybridization chamber as cited in the literature is the usually poor stable position of the humid chamber in the incubation oven. This poor stable position may lead to destabilization of the slide and to mixing of the solutions of the different slide wells.

Furthermore, the slide has to be rinsed firstly during the washing step and then has to be transferred to another container. In this relatively tedious process, unspecific binding of nucleic acid probe molecules to the cells may occur, due to decreased hybridization temperatures.

Other problems of the conventional FISH method are that during the washing step with distilled water, cells may be washed off or may be washed into another slide well, and that during air-drying in vertical position, cells may be transferred from one slide well to the next via drops that run down the slide. Due to the unstable positioning of the slide it may tip over, and the cells may be detached.

The poor reproducibility and the elaborate, tedious and inconvenient handling have led to rare use of the in situ hybridization in general and especially the FISH analysis in industry until now. However, since the analysis of bacteria using these procedures has significant advantages compared to all other microbiological analysis methods currently used in industry, there is a need for a device or a method which renders possible a simple and reproducible identification of microorganisms by in situ hybridization and especially by FISH procedure.

### SUMMARY OF THE INVENTION

It is thus an object of the present invention to overcome the above described disadvantages of the state of the art and to provide a device or an arrangement as well as a method by which fast identification of microorganisms in a sample can be performed easily and reproducibly.

Further objectives arise from the following description of the invention. The above mentioned objectives are solved according to the invention by the features of the independent claims. Further embodiments result from the features of the dependent claims.

### BRIEF DESCRIPTION OF THE DRAWINGS

FIG. 1. Illustration of a secondary structure model of the 16S rRNA.

FIG. 2. Schematic illustration of the FISH technique. During in situ hybridization, probes A and B, which are labeled differently, penetrate the cells A and C. The cell A contains ribosomal nucleic acids with the binding sites for probes of type A but not for probes of type B, and therefore probes of type B can not bind. Cell C does not contain binding sites for probe A nor probe B and can therefore bind neither of the two probes. After the subsequent washing step, only bound probes are present in the cell. Cell A can now be detected in the fluorescence microscope due to its fluorescence signal.

FIG. 3. Top plan view of the components of a special embodiment of the in situ hybridization arrangement according to the invention: container 1, tray 3, slide 4, lid 6 having supporting leg 8 (from left to right).

FIG. 4. Schematic illustration of an especially preferred embodiment of the lid 6, provided with slot 5 for fastening of the slide 4 and supporting leg 8 as well as the slide 4.

FIG. 5. Schematic illustration of a preferred embodiment of the in situ hybridization arrangement according to the invention. The tray 3 has little wells for uptake of liquid and is initially only partly inserted into the chamber 1 so that the tray 3 can be charged with the hybridization solution which is required for the humid chamber.

FIG. 6. Schematic illustration of a special embodiment of the in situ hybridization arrangement according to the invention with fully inserted tray 3.

FIG. 7. Schematic illustration of an especially preferred embodiment of the in situ hybridization arrangement according to the invention, in which the slide 4 fixed to a lid 6 is plugged in.

FIG. 8. Schematic illustration of a preferred embodiment of the in situ hybridization arrangement according to the invention. Lateral bearings or guide rails inside the chamber allow an easy insertion and further fixation or stabilization of the slide as well of the tray in the chamber. The chamber and the lid have a construction to allow a stable horizontal as well as vertical position.

a) Schematic perspective view in which parts of the arrangement are shown transparently for better understanding.

b) Outline illustration of a).

FIG. 9. Schematic illustration of the assembly of the individual components of the in situ hybridization arrangement according to the invention for specific detection of microorganisms by in situ hybridization using the in situ hybridization arrangement according to the invention.

FIG. 10. Scale drawing of a preferred embodiment of the lid 6.

FIG. 11. Scale drawing of a preferred embodiment of the tray 3.

FIG. 12. Scale drawing of a preferred embodiment of the container for the chamber 1.

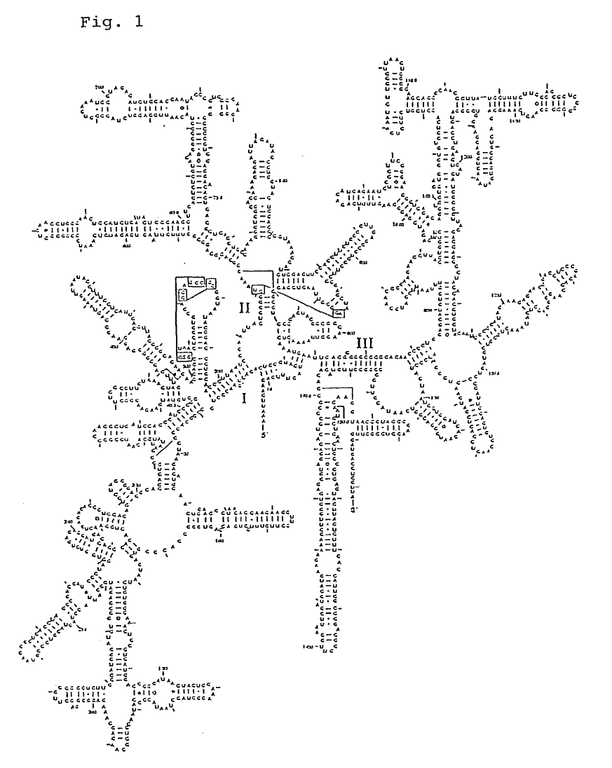

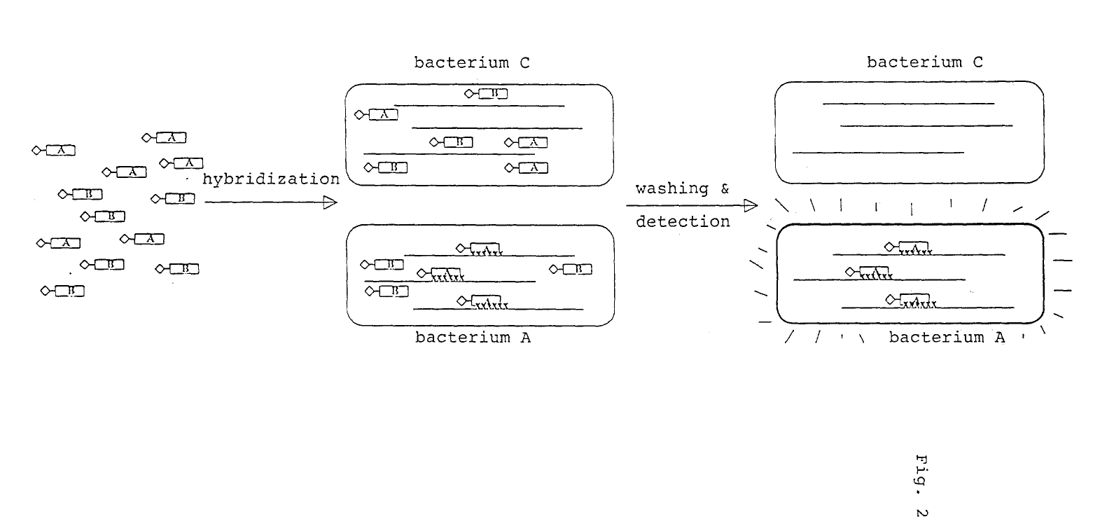

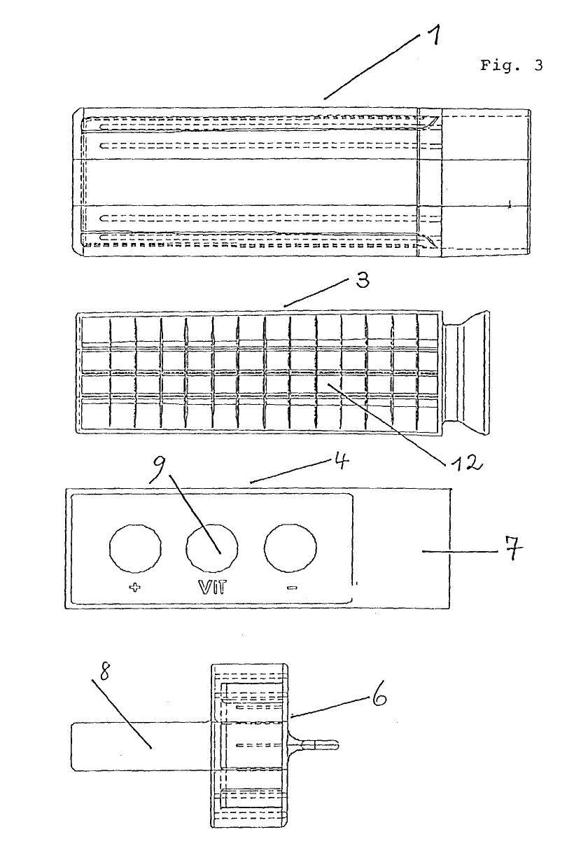

### DETAILED DESCRIPTION OF THE PREFERRED EMBODIMENT

According to the invention, an in situ hybridization arrangement for specific detection of microorganisms is provided, comprising a container 1 having at least one opening 2; a support for the hybridization solution 3; a slide 4 and a fastening means 5 for the slide. Preferably, the arrangement comprises a lid 6 suitable for tight sealing especially for water and/or airtight sealing of the opening of the container. The term “tight” in this context means that moisture present in the container essentially does not escape from the container when sealed.

The slide is provided preferably with wells 9 in which the sample to be analyzed and, optionally, negative or positive samples can be applied separately from each other. Especially preferably, the wells on the slide are adjacent to other wells only in one dimension, and are, for example, arranged in a row, wherein the wells may also be arranged in zigzag within the row.

In a preferred embodiment of the in situ hybridization arrangement according to the invention, the lid comprises the fastening means 5 for the slide. Especially preferably, the lid comprises a slot 5 as fastening means for the slide, in which one end 7 of the slide can be plugged in.

Alternatively, the slide can of course also engage with fastening means 5 which are components of the container and are for example in the form of a slot in the bottom of the container, wherein in this case the bottom is opposed to the opening of the container.

In another preferred embodiment of the arrangement according to the invention, the lid is provided with a structural element 8, which allows a stable position of the lid with fixed slide separate from the arrangement without lid when the slide is in a horizontal position.

Especially preferably, the lid is constructed in such a way that it allows the lid with the fixed slide to stand, separate from the arrangement without the lid when the slide is in a horizontal or vertical or lateral position.

Horizontal position of the slide within the scope of the present invention means that the position of the slide is such that the samples or probes may be applied onto the slide without the sample or probe flowing apart.

Lateral position of the slide within the scope of the present invention means a position rotated by 90° compared to the horizontal position, with the slide being rotated by 90° in such a way that drops could run off the slide, optionally without running into another well on the slide, provided that the wells are adjacent to other wells in only one dimension.

Vertical position of the slide within the scope of the present invention means a position that is rotated by 90° compared to the horizontal as well as to the lateral position.

In an especially preferred embodiment of the arrangement according to the invention, the container and/or the lid are constructed in such a way that when the lid is not closed as well as when the lid is closed a stable position of the arrangement is possible when the slide is arranged horizontally, vertically or laterally.

Preferred according to the invention is further an arrangement, in which the container is equipped with lateral bearings 10 or guide rails for the slide in order to stabilize the slide in the container or an arrangement, in which such bearings 10 are components of the container.

In addition, the hybridization solution support 3 is preferably removable or can be inserted. Preferred according to the invention is further that the support for the hybridization solution 3 can be inserted completely into the container. However, the support for the hybridization solution 3 can preferably also be inserted stepwise, especially continuously, into the container 1.

In another preferred embodiment, the container 1 is equipped with lateral bearings 11 for the hybridization solution support 3 in order to stabilize the hybridization solution support 3 in the container 1 or such bearings 11 are a component of the container 1.

Alternatively, the support for the hybridization solution is a fixed component of the container, especially a well or a recess in the container.

In an especially preferred embodiment of the arrangement according to the invention, the support for the hybridization solution is a tray 3, especially a tray provided with wells 12 for the uptake of liquid and/or for the uptake of liquid-soaked pads.

The materials for all components of the arrangement, except for the slide, preferably comprise plastics, especially preferred polyethylene and/or polypropylene. Furthermore, the materials for the before mentioned components of the arrangement may also comprise metals.

The slide is preferably made of glass, especially preferably of glass corresponding to the hydrolytic classes 1 to 4 according to DIN 12111.

In a further aspect of the present invention, a method for specific detection of microorganisms by in situ hybridization is provided comprising the following steps:

a) Fixing the microorganisms contained in a sample,

b) Incubating the fixed cells with detectable nucleic acid probe molecules,

c) Removing or washing-off the non-hybridized nucleic acid probe molecules, and

d) Detecting the cells hybridized with the nucleic acid probe molecules,

wherein steps a) to c) and, optionally, d) are carried out with the in situ hybridization arrangement according to the invention.

Preferably, the fixation and/or, optionally, the drying steps are carried out on the slide.

It is furthermore preferred according to the invention that the final drying of the slide is carried out when the slide is in a lateral position and/or the incubation is carried out when the slide is in a horizontal position and/or the washing is carried out when the slide is in a vertical position.

In another preferred embodiment of the method according to the invention, a mixture of a hybridization solution and a nucleic acid probe molecule solution is applied to the slide in step b).

Especially preferably, the mixture mentioned above is applied using a dropping vessel. This dropping vessel is in another preferred embodiment a single-use dropping vessel or a dropping vessel for multiple use.

In accordance to the method of the present invention, the hybridization solution, which is required for the humid chamber, can be filled into the arrangement of this invention through pads soaked with hybridization solution, which are located in the support for the hybridization solution.

Preferably, the nucleic acid probe molecule used in step b) is complementary to the chromosomal or an episomal DNA, an mRNA or an rRNA of a microorganism to be detected.

According to the invention it is further preferred that the nucleic acid probe molecule is covalently linked to a detectable marker. This detectable marker is preferably selected from the group of the following markers:

fluorescence marker,

chemoluminescence marker,

radioactive marker,

enzymatically active group,

hapten,

nucleic acid detectable by hybridization.

The microorganism in the method according to the invention is preferably a single-celled microorganism. Especially preferably, the microorganism is a yeast, a bacterium, an alga or a fungus. In another preferred embodiment, the microorganism is a wastewater bacterium.

In further embodiments of the method according to the invention, the sample is an environmental sample and taken from water, soil or air; or a food sample, particularly from milk or dairy products, drinking water, beverages, bakery products or meat products; or a medical sample, particularly a sample obtained from tissue, secreta or feces; or a waste water sample, particularly a sample obtained from activated sludge, digested sludge or anaerobic sludge; or a sample obtained from a biofilm, particularly a sample for which the biofilm is obtained from an industrial plant, is generated in the course of waste water treatment, or is a natural biofilm; or a sample taken from a pharmaceutical or cosmetic product.

Furthermore, according to the invention, a kit is provided for specific detection of micro-organisms by in situ hybridization, which comprises at least one nucleic acid probe molecule for specific detection of a microorganism; at least one hybridization solution; optionally, a nucleic acid probe molecule for performing a negative control; optionally, a nucleic acid probe molecule for performing a positive control; optionally, a washing solution, optionally, a fixation solution, optionally an antifading reagent as well as an in situ hybridization arrangement according to the invention.

The nucleic acid probe molecule in the kit according to the invention is preferably complementary to a chromosomal or an episomal DNA, an mRNA or an rRNA of a microorganism to be detected.

Preferred according to the invention is that the nucleic acid probe molecule in the kit according to the invention is preferably covalently linked to a detectable marker. Especially preferably, the detectable marker is selected from the group consisting of fluorescence markers, chemiluminescence markers, radioactive markers, enzymatically active groups, haptens and nucleic acids detectable by hybridization.

Another subject of the present invention is the use of the in situ hybridization arrangement according to the invention for specific detection of microorganisms by in situ hybridization.

Finally, another subject of the present invention is the use of the kit according to the invention in the method according to the invention.

It has now surprisingly become possible to provide an in situ hybridization arrangement that allows a fast and safe identification of microorganisms in a simple and reproducible way. The arrangement according to the invention comprises a container provided with at least one opening, which in the following is also designated as chamber; a support for the hybridization solution, especially a tray, which can be fully inserted into the container or chamber or which is part of the container; as well as a fastening means for a slide. In addition, the arrangement comprises a slide, which can be inserted in the chamber for in situ hybridization. The construction of an arrangement according to the invention is shown in FIG. 3.

Preferably, the arrangement according to the invention further comprises a lid suitable for tight sealing, especially for watertight and/or air tight sealing, of an opening of the container. The slide is preferably affixed to this lid, especially preferably it is plugged in. In this preferred embodiment, the slide is inserted tightly and safely in the lid but can be removed again manually and without excessive force from the lid for final analysis. The fixation or fastening of the lid makes it possible to conduct the washing procedure securely even in a vertical position of the arrangement according to the invention.

In an especially preferred embodiment of the arrangement according to the invention, the lid is provided with a structural part or the lid comprises a structural part, which allows a stable position of the lid with the fixed slide separate from the arrangement when the slide is in a horizontal or vertical position. This stability of the lid, which is obtained according to the invention for example by fitting the lid with a supporting leg, makes it possible to maintain the slide in a horizontal position during all reactions that take place.

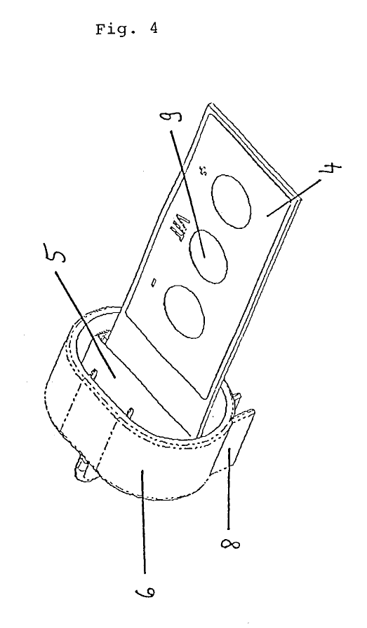

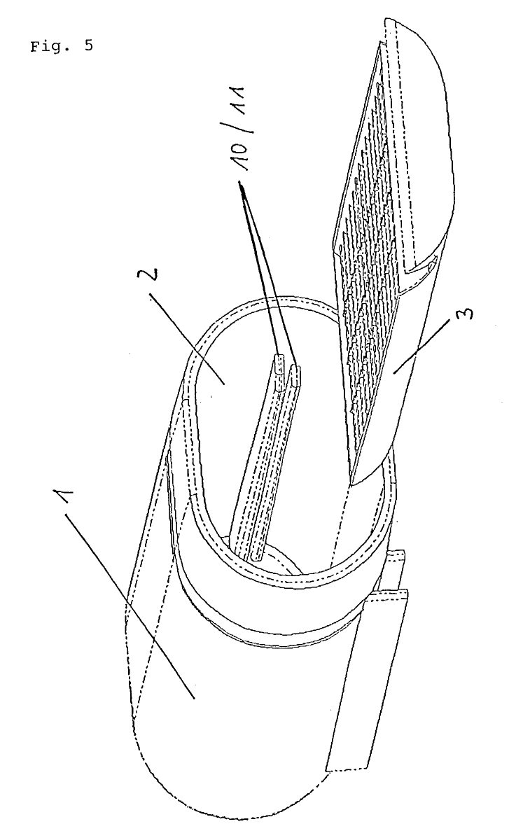

For easier handling the slide is preferably affixed to the lid of the in situ hybridization arrangement (see FIG. 4). In this case, the slide can remain affixed to the lid throughout the entire method of hybridization according to the invention. Furthermore, all preparative procedures such as washing, fixation and the like are feasible in the same reaction chamber.

Providing the lid with a structural part which allows a stable position of the lid separate from the arrangement when the slide is in a horizontal or vertical position, has the essential advantage that even during application of the samples and probes, the slide can be left in the lid, and at the same time, an even and secure position is provided during application of the samples.

Furthermore, the structural part or the supporting leg of the lid makes it possible to perform the individual reactions for achieving hybridization of nucleic acid probes with cells on the slide when the slide is fastened to the lid. Furthermore, all drying steps can also be conducted outside of the chamber with a lid that is provided with such a structural part. Due to the even and secure stand of the lid containing the fixed slide, mixing of the samples on the slide can be prevented.

In another preferred embodiment of the present invention, the container and/or the lid are constructed, so that when the lid is closed a stable position of the arrangement is possible when the slide is in a horizontal position. The horizontal position of the slide, especially during steps a) and b) of the method, is thus provided by the construction of the bearing surfaces of the components of the in situ hybridization arrangement according to the invention.

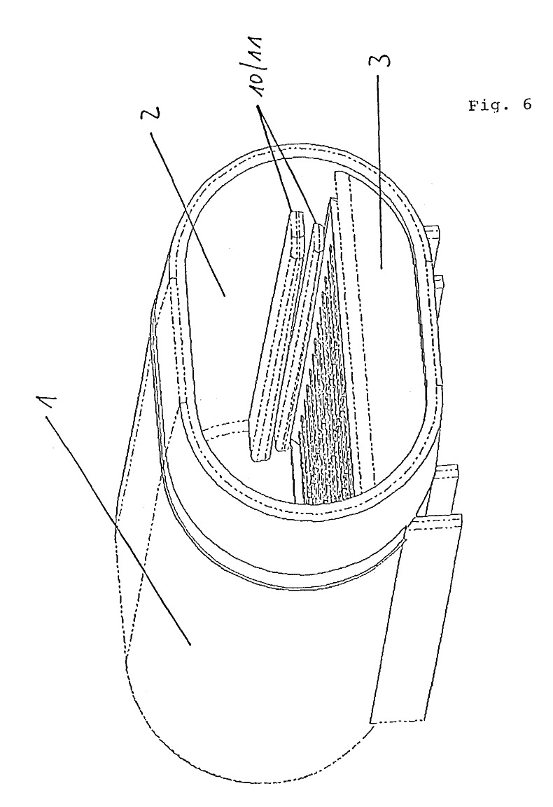

Using a tray as support for the hybridization solution makes it possible to insert the hybridization solution, which is required for the humid chamber safely and cleanly in the arrangement according to the invention. The tray preferably has small wells or recesses for the uptake of liquid (see FIG. 5). Especially preferred is that initially the tray is only partly inserted into the chamber, so that the hybridization solution which is required for providing a humid chamber, can be filled into the tray. Then, the tray preferably is completely inserted into the chamber (see FIG. 6).

Alternatively, cellulose may be used as support for the hybridization solution.

The construction of an exchangeable tray into which the hybridization solution may be dropped, and which can be removed after use, has the advantage that fast introduction of a defined amount of hybridization solution in the in situ hybridization arrangement according to the invention is possible. The use of cellulose is not required but is not excluded either.

Another alternative for introduction of the hybridization solution in the in situ hybridization arrangement according to the invention is to introduce the hybridization solution in the reactor through single-use pads, which are located in the tray. Preferably, the single-use pads are sealed with a fresh-keeping seal, which is removed as soon as the tray is in the chamber, and the hybridization solution then can evaporate in the chamber.

A preferred embodiment for the method carried out in the in situ hybridization arrangement according to the invention for fast and simple practice of the in situ hybridization for the specific analysis of microorganisms comprises the following steps:

a) fixing the microorganisms contained in a sample on a slide;

b) incubating the fixed cells with nucleic acid probes in the arrangement according to the invention in order to achieve hybridization;

c) removing or washing-off the non-hybridized oligonucleotides;

d) detecting the cells hybridized to the oligonucleotides.

Incubation and washing procedure preferably take place in the in situ hybridization arrangement according to the invention.

Preferably, the microorganisms are not fixed first in a reaction vessel and then immobilized, as it is usually done, but the fixation and/or, optionally, the drying take place directly on the slide.

Such a fixation on the slide avoids cell losses, and its handling is significantly easier and much less complicated in practice. In addition, the fixation on the slide allows combination of the fixation step and the dehydration series in one procedure.

The hybridization and addition of the nucleic acid probe molecules is according to the invention preferably not performed by pipetting first a defined amount of hybridization solution and then a defined amount of probe solution into a slide well using a pipette, as it is usually done, but by applying a mixture of a hybridization and a nucleotide probe molecule solution onto the slide.

The application of the mixture of hybridization and nucleic acid probe molecule solution allows a faster and flawless procedure when the nucleic acid probe molecules are comparably stable.

The above mentioned mixture is preferably applied dropwise by applying light pressure to a dropping vessel. The dropping vessel may be intended for multiple use and may contain several drops of the mixed solution of hybridization and probe solutions, or it may alternatively be a small single-use dropping vessel which contains the required quantity of reagents having regard to a dead volume.

The use of dropping vessels eliminates the use of expensive pipettes and in addition facilitates handling and dosage.

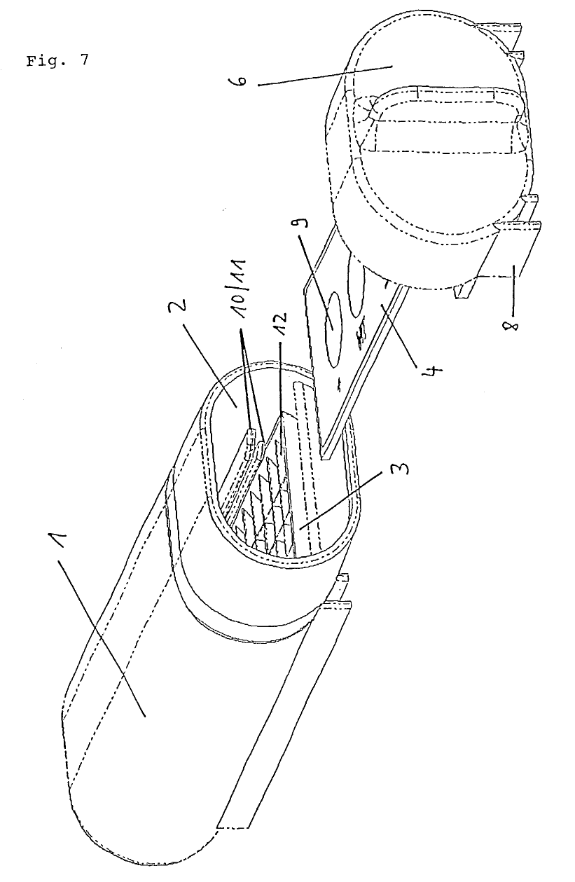

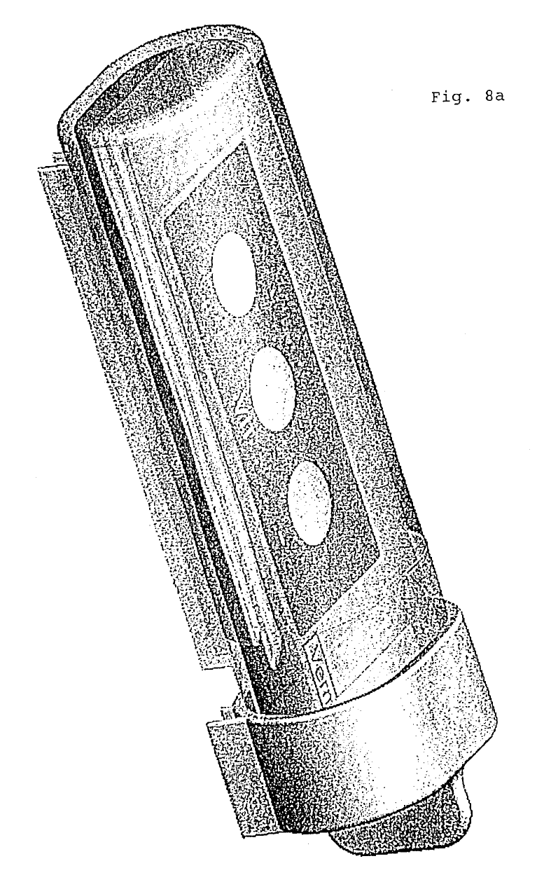

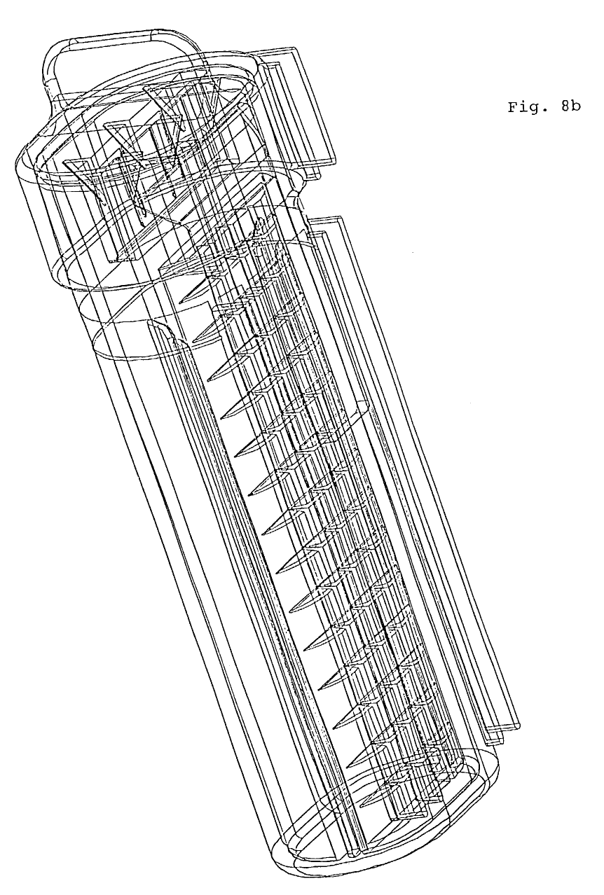

Then, the slide preferably fastened to the lid is inserted into the chamber (see FIG. 7). Lateral bearings or guide rails are preferably affixed inside the chamber in order to provide easy insertion and further fixation or stabilization of the slide in the chamber (see FIG. 8).

Preferably, the chamber and/or the lid are constructed in such way that a stable horizontal as well as vertical or lateral position is ensured.

The subsequent incubation is performed preferably in the horizontal position of the slide. The subsequent washing of the slide is performed according to the invention preferably in the chamber and especially preferably with the slide being positioned vertically.

For final drying and especially for final air drying of the slide, the lid is preferably constructed in such way that it can be positioned laterally. This is especially advantageous since the drops can run down the slide without running into another well of the slide.

As a result, most of the procedure steps are preferably conducted in a single vessel consisting of a chamber and preferably a lid of the arrangement.

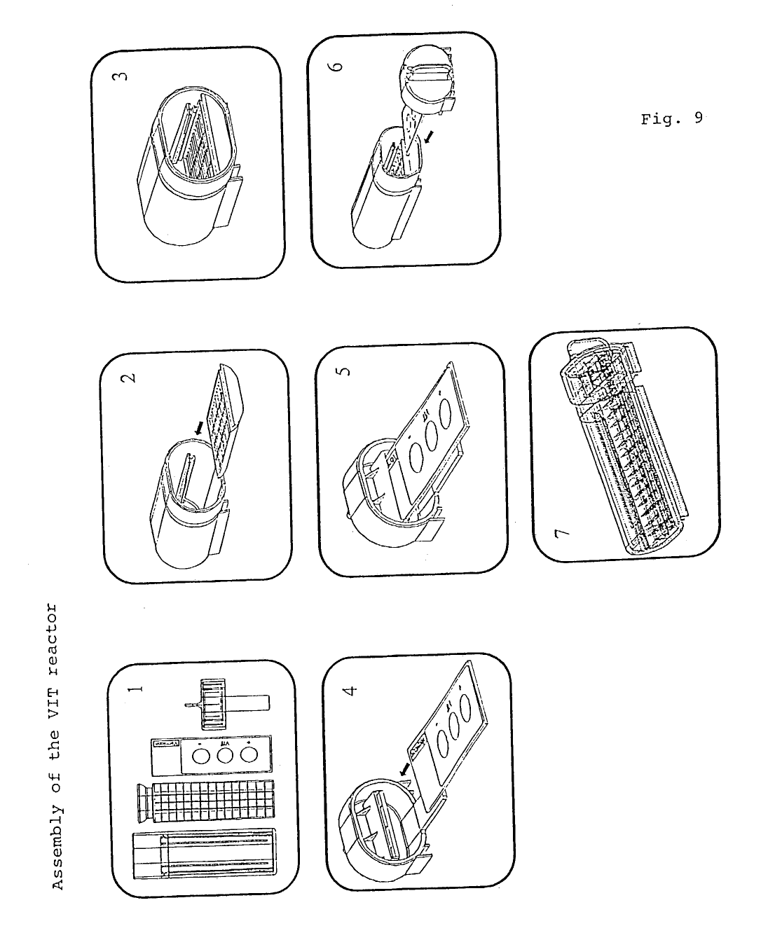

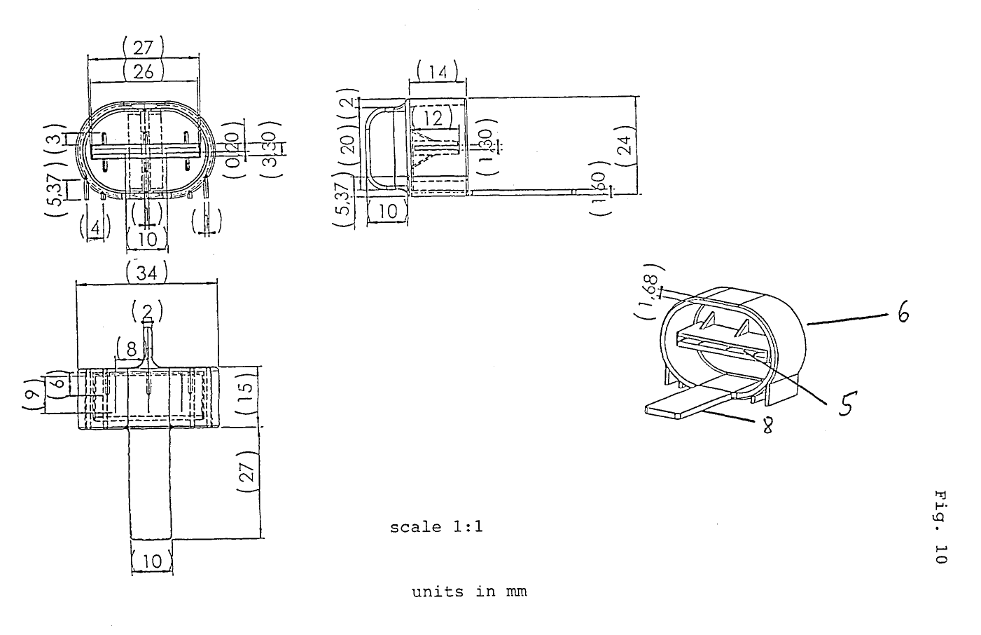

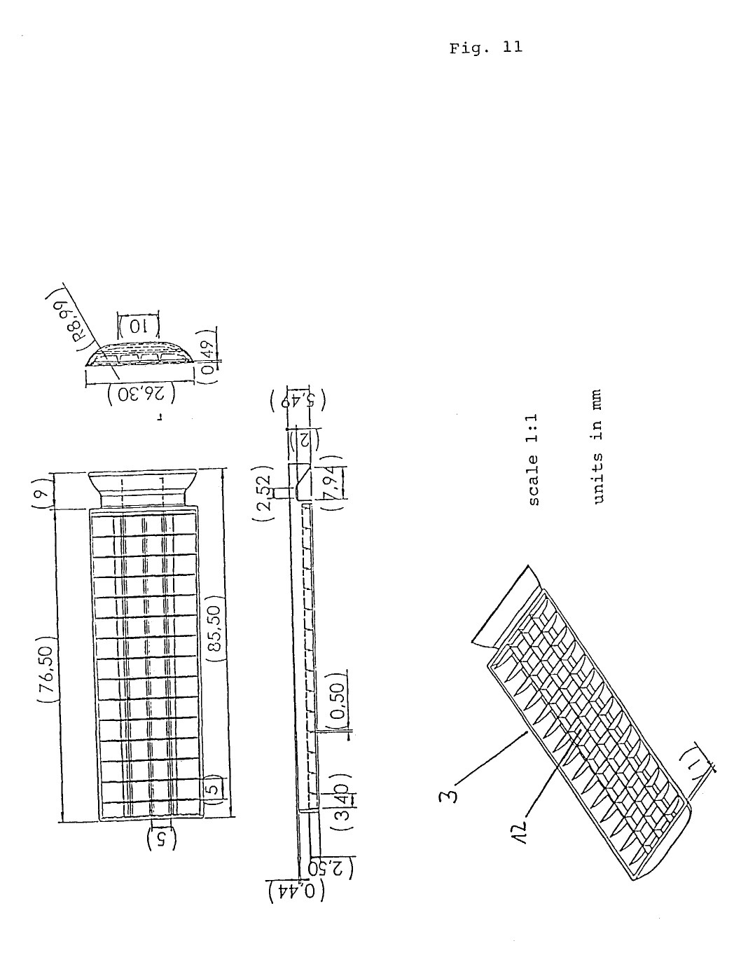

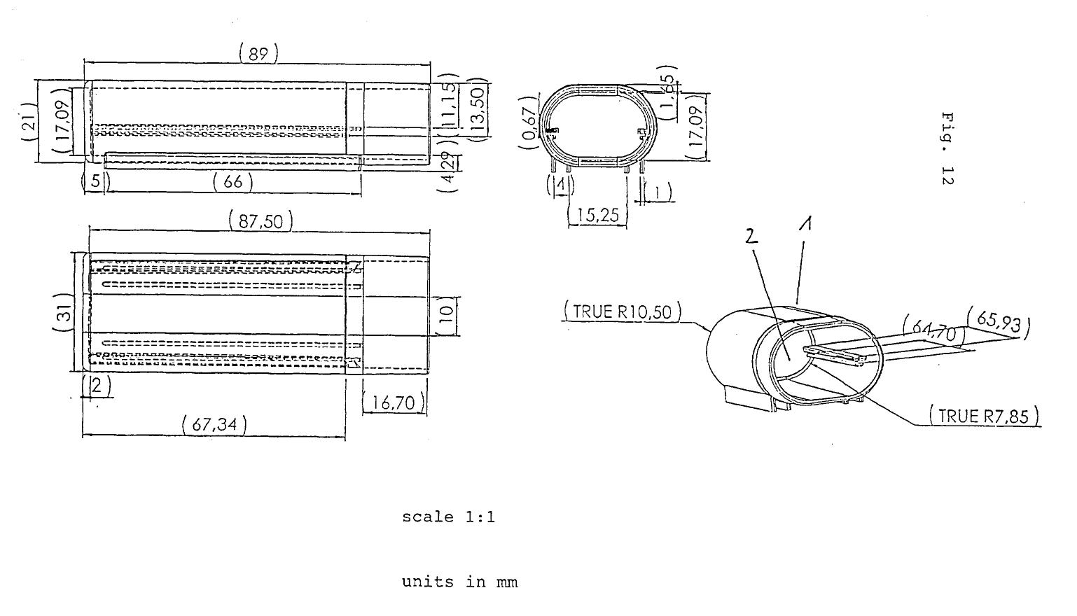

In FIG. 9, the entire construction of a preferred embodiment of the in situ hybridization arrangement according to the invention, comprising lid, chamber, tray and inserted slide, is shown. FIGS. 10 to 12 show the dimensions of lid, tray and chamber.

Essential advantages of the in situ hybridization arrangement according to the invention and the method according to the invention for specific detection of microorganisms compared to conventional methods for in situ hybridization and especially for conventional FISH methods are therefore the very easy handling as well as the speed and reproducibility with which the specific detection of microorganisms in a sample is made possible.

Within the scope of the present invention, “fixation” of microorganisms is meant to be a treatment, with which the cell envelope of the microorganisms is made permeable for nucleic acid probes. The nucleic acid probes, consisting of an oligonucleotide and a marker linked thereto, are then able to penetrate the cell envelope in order to bind to the target sequence that corresponds to the nucleic acid probe inside the cell. The binding is to be understood as a formation of hydrogen bonds among complementary nucleic acid regions. The envelope can be a lipid envelope coating a virus, the cell wall of bacteria or the cell membrane of a single-celled eukaryote. For fixation, usually ethanol is used. If the cell wall cannot be made permeable for nucleic acid probes with these measures, the expert will know further measures that lead to the same result. These include for example a low-percentage paraformaldehyde solution or a diluted formaldehyde solution, methanol, alcohol mixtures, enzymatic treatments or the like.

The nucleic acid probe in the sense of the invention may be a DNA or an RNA probe, usually comprising between 12 and 1000 nucleotides, preferably between 12 and 50, especially preferred between 17 and 25 nucleotides. The selection of the nucleic acid probes is performed according to the criteria of whether a complementary sequence is present in the microorganism to be detected. By selecting a defined sequence, a bacterial species, bacterial genus or an entire bacterial group can be detected. In a probe having a length of 12 to 15 nucleotides, 100% of the sequence must be complementary. In oligonucleotides with more than 15 nucleotides, one to several mismatches are permitted. In compliance with stringent hybridization conditions it is provided that the nucleic acid probe molecule in fact hybridizes to the target sequence. Moderate conditions in the sense of the invention are e.g. 0% formamide in a hybridization solution as described in Example 1. Stringent conditions in the sense of the invention are for example 20-80% formamide in the hybridization solution.

The duration of the hybridization usually is between 10 minutes and 12 hours; preferably the hybridization lasts for approximately 2 hours. The hybridization temperature is preferably between 44° C. and 48° C., especially preferably 46° C., wherein the parameter of the hybridization temperature as well as the concentration of salts and detergents in the hybridization solution may be optimized depending on the probe or the probes, especially their length(s) and the degree of complementarity to the target sequence in the cell to be detected. Calculations that are typical here are known to the person skilled in the art.

Within the scope of the method according to the invention, a typical hybridization solution has a salt concentration of 0.1 to 1.5 M, preferably of 0.9 M, with the salt being preferably sodium chloride. Further, the hybridization solution usually comprises a detergent such as e.g., sodium dodecylsulfate (SDS), in a concentration of 0.001-0.1%, preferably in a concentration of 0.01%, and Tris/HCl in a concentration ranging from 0.001-0.1 M, preferably in a concentration of 0.02 M. The pH of Tris/HCl is usually between 6 and 10, although a pH of approximately 8.0 is preferred. As mentioned above, the hybridization solution may further contain between 0% and 80% formamide, depending on which degree of stringency is desired or required.

The nucleic acid probe should be present in the hybridization solution, if possible, in a quantity of 15 ng to 1000 ng, wherein this amount should be contained in a hybridization solution volume between 8 μl and 106 μl, preferably in 40 μl. Especially preferred, the probe concentration is 111 ng/40 μl hybridization solution.

After the hybridization has been finished, the non-hybridized and excessive probe molecules should be removed, which usually is performed using a conventional washing solution or a conventional washing buffer. This washing solution may contain, if desired, 0.001-0.1% of a detergent such as SDS, wherein a concentration of 0.01% is preferred, as well as Tris/HCl in a concentration of 0.001-0.1 M, preferably 0.02 M, with the pH of Tris/HCl being in the range of 6.0 to 10.0, preferably 8.0. A detergent may be present, but this is not an absolute requirement. The washing solution usually further contains NaCl, the concentration being 0.003 M to 0.9 M, preferably 0.01 M to 0.9 M, depending on the required stringency. Furthermore the washing solution may contain EDTA, the concentration being preferably 0.005 M. The washing solution may further contain usual preservatives known to the person skilled in the art, in suitable amounts.

The “washing-off” of the unbound probe molecules usually is performed at a temperature in the range of 44° C. to 52° C., preferably of 44° C. to 50° C. and especially preferred at 46° C. for a period of 10-40 minutes, preferably for 15 minutes.

The selection of the respective nucleic acid probes is based on the microorganism to be detected. The nucleic acid probe may hereby be complementary to a chromosomal or an episomal DNA, but also to an mRNA or an rRNA of the microorganism to be detected. It is advantageous to select a nucleic acid probe that is complementary to a region, which is present in a copy number of more than 1 in the microorganism to be detected. The sequence to be detected preferably is present in a copy number of 500-100000 per cell, especially preferably in copy number of 1000-50000. For this reason, the rRNA is used preferably as target site, since the ribosomes of the cell are the sites of protein biosynthesis and are present in many thousand copies in each active cell.

According to the invention, the nucleic acid probe is incubated with the microorganism that has been fixed in the above sense, in order to allow penetration of the nucleic acid probe molecules into the microorganism and the hybridization of nucleic acid probe molecules with the nucleic acids of the microorganism. Then, the non-hybridized nucleic acid probe molecules are removed by usual washing steps. The specifically hybridized nucleic acid probe molecules then can be detected in the respective cells.

A prerequisite for the identification and for the quantification is that the nucleic acid probe molecule that is used according to the invention is detectable. This detectability may be provided e.g., by a covalent linkage of the nucleic acid probe molecule to a detectable marker. As detectable markers, fluorescent groups such as e.g. CY2, CY3, CY5, FITC, FLUOS, TRITC, or FLUOS-PRIME are used which are all well known to the expert. Examples for fluorescent groups are listed in the following Table 1.

TABLE 1

| | |
|---|---|
| FLUOS: | 5,(6)-carboxyfluorescein-N-hydroxysuccinimide ester |
| | (Boehringer Mannheim, Mannheim, Germany); |
| | ε = 7.50 × 104 mol⁻¹ l⁻¹, absₘₐₓ at 494 nm; |
| | Emₘₐₓ at 518 nm, MW = 473. |
| TRITC: | tetramethylrhodamine-5,6-isothiocyanate |
| | (Isomer G. Molecular Probes Inc., Eugene, USA, Lambda, |
| | Graz, AT); ε = 1.07 × 105 mol⁻¹ l⁻¹, absₘₐₓ at 537 nm; |
| | Emₘₐₓ at 566 nm, MW = 479. |
| CT: | 5,(6)-carboxytetramethylrhodamine-N-hydroxysuccinimide |
| | ester (Molecular Probes Inc., Eugene, USA); |
| | ε = 0.87 × 105 mol⁻¹ l⁻¹, absₘₐₓ at 537 nm; |
| | Emₘₐₓ at 566 nm. |
| CY-3: | NHS ester of Cy5.18 (Biological Detection Systems, |
| | Pittsburgh, USA); (Amersham Life Sciences, Inc., Arlington |
| | Heights, USA); ε = 1.5 × 105 mol⁻¹ l⁻¹, absₘₐₓ at 532 nm; |
| | Emₘₐₓ at 565 nm. MW = 765.95. |
| CY-5: | NHS ester of Cy5.18 (Biological Detection Systems, |
| | Pittsburgh, USA); (Amersham Life Sciences, Inc., Arlington |
| | Heights, USA); ε => 2 × 105 mol⁻¹ l⁻¹, absₘₐₓ at 650 nm; |
| | Emₘₐₓ at 667 nm. MW = 791.99. |

Alternatively, chemiluminescent groups or radioactive labels such as ³⁵S, ³²P, ³³P, ¹²⁵I are used. However, detectability may also be provided by coupling of the nucleic acid probe molecule with an enzymatically active molecule, e.g. alkaline phosphatase, acid phosphatase, peroxidase, horseradish peroxidase, β-D-galactosidase, or glucose oxidase. For each of these enzymes, a number of chromogens is known which can be transformed instead of the natural substrate, and which can be transformed to colored or fluorescent products. Examples of such chromogens are given in the following Table 2.

TABLE 2

| Enzymes | Chromogen |
|---|---|
| 1. Alkaline | 4-methylumbelliferyl phosphate (*), bis(4-methyl- |
| phosphatase | umbelliferyl phosphate), (*) 3-O-methylfluorescein, |
| and acid | flavone-3-diphosphate triammonium salt (*), p-nitro- |
| phosphatase | phenylphosphate disodium salt. |
| 2. Peroxidase | tyramine hydrochloride (*), 3-(p-hydroxyphenyl)- |
| | propionic acid (*), p-hydroxyphenethyl alcohol (*), 2,2'- |
| | azino-di-3-ethylbenzthiazoline sulfonic acid (ABTS), |
| | ortho-phenylendiamine dihydrochloride, o-dianisidine, |
| | 5-aminosalicylic acid, p-ucresol (*), 3,3′-dimethyloxy |
| | benzidine, 3-methyl-2-benzothiazoline hydrazone, |
| | tetramethylbenzidine |
| 3. Horseradish | H₂O₂ + diammonium benzidine |
| peroxidase | H₂O₂ + tetramethylbenzidine |
| 4. β-D- | o-nitrophenyl-β-D-galactopyranoside, 4-methyl- |
| galactosidase | umbelliferyl-β-D-galactoside |
| 5. Glucose | ABTS, glucose and thiazolyl blue. |
| oxidase | |

*fluorescence

Finally it is possible to create nucleic acid probe molecules in such a way that they have another nucleic acid sequence at their 5′ or 3′ end that is suitable for hybridization. This nucleic acid sequence again comprises approx. 12 to 1000, preferably 15-50 nucleotides. This second nucleic acid part can again be recognized by an oligonucleotide probe detectable by any of the above mentioned compounds or agents.

Another possibility is the coupling of the detectable nucleic acid probe molecules with a hapten. After detaching the nucleic acid probe molecules from the target nucleic acid, the nucleic acid probe molecules, which are now present separately, can be contacted with detectable antibodies recognizing the hapten. A well known example of such a hapten is digoxigenin or its derivatives. The person skilled in the art knows many other possibilities apart from the here mentioned examples to detect and to quantify an oligonucleotide used for hybridization.

The multitude of possible labels further allows the simultaneous detection of two or more overlapping or non-overlapping populations. Thus, for example by using two or more different fluorescence markers, several bacterial communities may be detected (R. Amann, J. Snaidr, M. Wagner, W. Ludwig, and K.-H. Schleifer, In situ visualization of high genetic diversity in a natural microbial community, J. Bacteriol. (1996) 178:12,3496-3500).

The evaluation depends on the kind of labeling of the used probe. Within the scope of the present invention, the evaluation can be performed advantageously by a light-optical microscope, epifluorescence microscope, chemiluminometer, fluorometer and the like.

The microorganism to be detected using the method according to the invention can be a prokaryotic or eukaryotic microorganism. In most cases it may be desired to detect single-celled microorganisms. These single-celled microorganisms may also be present in larger aggregates, the so-called filaments. Relevant microorganisms are hereby primarily yeasts, algae, bacteria or fungi.

In an especially preferred embodiment of the present invention, the microorganisms are bacteria, which are present in the waste water of waster water treatment plants.

The method according to the invention may be used manifold. Environmental samples can be analyzed for the presence of microorganisms. For this, these samples can be taken from air, water or soil.

Another field of application for the method according to the invention is the control of food articles. In preferred embodiments, the food samples are taken from milk or dairy products (yogurt, cheese, cottage cheese, butter, buttermilk), drinking water, beverages (lemonades, beer, juices), bakery products or meat products. For the detection of microorganisms in food, cultivation may be possible in some instances, to ensure that microorganisms are present in sufficient quantities.

The method according to the invention may further be used for analysis of medical samples. It is suited for the analysis of tissue samples such as biopsy material from the lungs, tumor or inflammatory tissues, from secreta such as sweat, saliva, semen and nasal secretions, urethra or vaginal discharges as well as for urine or stool samples.

A further field of application of the present method is the analysis of wastewater, e.g. activated sludge, digested sludge or anaerobic sludge. Furthermore, it is suited to analyze biofilms in industrial plants, and to analyze naturally forming biofilms, or biofilms being formed in the course of waste water treatment. The analysis of pharmaceutical and cosmetic products such as ointments, cremes, tinctures, liquid formulations, etc. is possible with the method according to the invention.

According to the invention, in a further aspect of the present invention, a kit for applying the method for detection of microorganisms in a sample is provided. The content of such a kit are based essentially upon the nature of the microorganism to be detected. It comprises as the main component one or more nucleic acid probe(s) specific for each of the microorganism to be detected, as well as preferably further nucleic acid probes with which a negative or positive control can be performed. Furthermore, it comprises preferably a hybridization solution and a washing solution. The selection of the hybridization solution primarily depends on the length of the used nucleic acid probes. Thus, as it is known to one skilled in the art, less stringent conditions must be selected for the hybridization of a nucleic acid probe of 15 nucleotides than for hybridization of a probe with a length of 75 nucleotides. Examples for hybridization conditions are given e.g., in Stahl & Amann (1991) in Stackebrand and Goodfellow (eds.), Nucleic Acid Techniques in Bacterial Systematics; John Wiley & Sons Ltd., Chichester, UK.

Thus, according to the invention, a kit is provided with which the above described method according to the invention can be conducted. The kit according to the invention comprises in a preferred embodiment at least one nucleic acid probe molecule for specific detection of a microorganism; at least one hybridization solution; optionally, a nucleic acid probe molecule for performing a negative control; optionally, a nucleic acid probe molecule for performing a positive control; optionally, a washing solution; optionally, a fixation solution; optionally, an anti-fading reagent; as well as the in situ hybridization arrangement according to the invention, with the following steps being conductible in the arrangement or in parts of the arrangement:

a) fixing the microorganisms contained in a sample on a slide;

b) incubating the fixed cells with nucleic acid probes to achieve hybridization;

c) removing or washing-off the non-hybridized nucleotide probe molecules.

In a preferred embodiment, the kit contains specific probes for detection of bacteria that are present in the waste water of wastewater treatment plants.

Using the method according to the invention, in situ hybridization can be established in practice.

The following Examples and Figures serve to describe the invention, and are not intended to be interpreted as to restrict the invention in any way.

### EXAMPLE 1

### Detection of Bacteria in a Waste Water Sample

1. General Description

The following example of the method according to the invention, in the following also named “VIT method”, in the in situ hybridization arrangement according to the invention, in the following also named “VIT reactor”, serves for the qualitative analysis of bacteria being present in waste water samples. The identification is completed within a few hours.

2. Basic Principle

In this procedure, the bacteria are hybridized with fluorescence-labeled oligonucleotide probes, and then can be detected on the slide in an epifluorescence microscope.

3. Materials

Drying cabinet, preheated to 46° C.

Bottle for preparing and heating the washing solution.

Graduated cylinder for preparation of the washing solution.

Thermometer.

Timer.

VIT solution: solution containing specific nucleic acid probe molecules.

Negative control: solution for negative control.

Positive control: solution for positive control.

Solutions A and B: fixation solutions.

Solution C: hybridization solution.

Solution D: washing solution.

Finisher: anti-fading reagent.

Slide having three wells (1 well for the actual hybridization, marked with “VIT”; 1 well for the negative control, marked with “−”; 1 well for the positive control, marked with “+”).

Coverslips.

4. Procedure

Preheat drying chamber to 46° C. prior to analysis.

Apply samples and fix them.

1. Plug in the slide into the lid of the VIT reactor.

2. Transfer 1 drop of sample material in each of the three wells on the slide, incubate slide (without VIT reactor) horizontally (46° C., 30 min, or until completely dry).

3. Apply 1 drop of “solution A” in each well on the slide, incubate slide (without VIT reactor) horizontally (46° C., 30 min, or until completely dry).

4. Apply 1 drop of “solution B” in each well on the slide, incubate slide (without VIT reactor) horizontally (room temperature, 1 min, or until completely dry).

Hybridization

5. Apply 1 drop of “negative control” onto the slide well marked with “−”.

6. Apply 1 drop of “positive control” onto the slide well marked with “+”.

7. Apply 1 drop of “VIT” onto the slide well marked with “VIT”.

8. Insert tray halfway into the VIT reactor.

9. Apply approx. 20-30 drops of “solution C” into the tray of the VIT reactor, insert tray fully into the reactor.

10. Insert slide carefully and horizontally into the VIT reactor, close VIT reactor and incubate horizontally (46° C., 1.5 h).

ATTENTION: The individual drops may NOT be allowed to mix!

11. Prepare washing solution.

25 ml washing solution are required for each VIT reactor. For this, “solution D” is diluted tenfold with distilled water. In Table 3, dilution examples are given.

TABLE 3

| | Quantities in ml for | | |
|---|---|---|---|
| | 1 VIT reactor | 3 VIT reactors | 10 VIT reactors |
| Solution D | 2.5 | 7.5 | 25 |
| Distilled water | 22.5 | 67.5 | 225 |
| Final volume | 25 | 75 | 250 |

12. Preheat the final washing solution in a closed vessel in the drying cabinet to 46° C. during hybridization.

13. Open VIT reactor carefully and remove slide.

14. ATTENTION: The individual drops may NOT be allowed to be mixed

15. Bring the VIT reactor in position and add preheated (see step 5.2.7) washing solution up to the graduation. Insert slide vertically; close VIT reactor and incubate vertically (46° C., 15 min).

16. Open VIT reactor and remove slide. Pour out washing solution.

17. Add distilled water to the VIT reactor up to the graduation. Insert slide vertically, and then remove it quickly. Pour out the water.

18. Bring the slide in a vertical position and incubate (46° C., 15 min or until completely dry).

19. Apply 1 drop of “finisher” each between the slide wells, and apply the coverslip.

What is claimed is:

1. An in situ hybridization arrangement for the specific detection of microorganisms in a sample, comprising:
a container having at least one opening,
a support for a hybridization solution,
a slide, and
a fastener for said slide.

2. The arrangement of claim 1, wherein said fastener is a lid, said lid is adapted for tightly sealing of said opening of said container.

3. The arrangement of claim 2, wherein said lid comprises fastening means for fastening said slide.

4. The arrangement of claim 3, wherein said fastening means for fastening said slide is a slot adapted for plugging in of said slide.

5. The arrangement of claim 2, wherein said lid comprises a structural part, which allows a stable position of said lid with said slide.

6. The arrangement of claim 5, wherein said stable position is a horizontal position.

7. The arrangement of claim 1, wherein said slide comprises at least one well.

8. The arrangement of claim 1, wherein said container comprises lateral bearings adapted to stabilize said slide in said container.

9. The arrangement of claim 1, wherein said support for the hybridization solution is removable.

10. The arrangement of claim 9, wherein said support for the hybridization solution is adapted to be inserted completely into said container.

11. The arrangement of claim 9, wherein said support for the hybridization solution is adapted to be inserted partially into said container.

12. The arrangement of claim 1, wherein said container comprises lateral bearings adapted to stabilize said support for the hybridization solution in said container.

13. The arrangement of claim 1, wherein said support for the hybridization solution is a fixed component of the container.

14. The arrangement of claim 13, wherein said support for the hybridization solution is a well in the container.

15. The arrangement of claim 1, wherein said support for the hybridization solution is a tray, wherein said tray comprises wells for a liquid or liquid-soaked pads.

16. The arrangement of claim 1, wherein said container, said support for hybridization solution and said fastener for said slide comprise plastics.

17. The arrangement of claim 16, wherein said plastics is polyethylene, polypropylene, or a combination thereof.

18. The arrangement of claim 1, wherein said slide is made of glass.

19. The arrangement of claim 18, wherein said glass is selected from the group consisting of hydrolytic classes 1 to 4 according to DIN 12111.

20. A method for specific detection of microorganisms in a sample, comprising the steps:
a) fixing the microorganisms contained in said sample,
b) incubating the fixed microorganisms with detectable nucleic acid probe molecules, thereby hybridizing said microorganisms to said detectable nucleic acid probe molecules,
c) removing non-hybridized nucleic acid probe molecules, and
d) detecting the microorganisms hybridized with the nucleic acid probe molecules,
wherein the steps a) to c) are carried out in the in situ hybridization arrangement of claim 1.

21. The method of claim 20, wherein step d) is carried out in the in situ hybridization arrangement of claim 1.

22. The method of claim 20, wherein said fixing is carried out on said slide.

23. The method of claim 20, wherein said removing is performed by washing off said non-hybridized nucleic acid probe molecules.

24. The method of claim 20, further comprising drying, wherein said drying is carried out when said slide is in a lateral position.

25. The method of claim 20, wherein said incubating is carried out when said slide is in a horizontal position.

26. The method of claim 23, wherein said washing is carried out when the slide is in a vertical position.

27. The method of claim 20, wherein in step b) a mixture of a hybridization solution and a nucleic acid probe molecule solution is applied to the slide.

28. The method of claim 27, wherein said mixture is applied using a dropping vessel.

29. The method of claim 28, wherein said dropping vessel is a single-use dropping vessel or a dropping vessel for multiple use.

30. The method of claim 27, wherein said hybridization solution is introduced into the arrangement through pads soaked with said hybridization solution, said pads are located in the support for the hybridization solution.

31. The method of claim 27, wherein said nucleic acid probe molecule solution comprises a nucleic acid probe molecule, said nucleic acid probe molecule is complementary to a chromosomal or an episomal DNA, to an mRNA or to an rRNA of a microorganism to be detected.

32. The method of claim 31, wherein said nucleic acid probe molecule is covalently linked to a detectable marker.

33. The method of claim 31, wherein said detectable marker is selected from the group consisting of: chemiluminescence marker, radioactive marker, enzymatically active group, hapten, and a nucleic acid detectable by hybridization.

34. The method of claim 20, wherein said microorganism is a single-celled microorganism.

35. The method of claim 20, wherein said microorganism is a yeast, a bacterium, an alga or a fungus.

36. The method of claim 35, wherein said microorganism is a wastewater bacterium.

37. The method of claim 20, wherein said sample is an environmental sample taken from water, soil or air.

38. The method of claim 20, wherein said sample is a food sample.

39. The method of claim 38, wherein said food sample is taken from dairy products, drinking water, beverages, bakery products or meat products.

40. The method of claim 20, wherein said sample is a medical sample.

41. The method of claim 40, wherein said medical sample is obtained from tissue, secreta or feces.

42. The method of claim 20, wherein said sample is obtained from wastewater.

43. The method of claim 42, wherein said sample is obtained from activated sludge, digested sludge or anaerobic sludge.

44. The method of claim 20, wherein said sample is obtained from a biofilm.

45. The method of claim 44, wherein said biofilm is obtained from an industrial plant, is generated in the course of a wastewater treatment, or is a natural biofilm.

46. The method of claims 20, wherein said sample is taken from a pharmaceutical or cosmetic product.

47. A kit for a specific detection of microorganisms by in situ hybridization, comprising:
at least one nucleic acid probe molecule for specific detection of a microorganism,
at least one hybridization solution, and
an in situ hybridization arrangement of claim 1.

48. The kit of claim 47, further comprising a nucleic acid probe molecule for performing a negative control.

49. The kit of claim 47, further comprising a nucleic acid probe molecule for performing a positive control.

50. The kit of claim 47, further comprising a washing solution.

51. The kit of claim 47, further comprising a fixation solution.

52. The kit of claim 47, wherein said nucleic acid probe molecule is complementary to a chromosomal or an episomal DNA, to an mRNA or to an rRNA of a microorganism to be detected.

53. The kit of claim 47, wherein said nucleic acid probe molecule is covalently linked to a detectable marker.

54. The kit of claim 53, wherein said detectable marker is selected from the group consisting of: fluorescence marker, chemiluminescence marker, radioactive marker, enzymatically active group, hapten, nucleic acid detectable by hybridization.
